(19) United States
(12) Patent Application Publication
(10) Pub. No.: US 2025/0118397 A1
(43) Pub. Date: Apr. 10, 2025

(54) SYSTEMS AND METHODS FOR A REAL-TIME FEEDBACK INTEGRATED CARE MANGEMENT PLATFORM

(71) Applicant: Somatus, Inc., McLean, VA (US)

(72) Inventors: Jorge Neyra, Germantown, MD (US); Eliot Davila, Laurel, MD (US); Bassem Mikhael, Arlington, VA (US)

(21) Appl. No.: 18/377,692

(22) Filed: Oct. 6, 2023

(51) Int. Cl. G16H 10/60; G06N 20/00; G06V 30/416; G16H 40/67; G16H 50/70

(52) U.S. Cl. CPC G16H 10/60; G06N 20/00; G16H 40/67; G16H 50/70; G06V 30/416

(57) ABSTRACT

A method and apparatus for a care management platform and a user device interacting during the providing of real-time provider intelligence information are described. The method includes receiving, from the user device, a request for patient records relevant to a patient, the request comprising a patient identifier (ID), a care provider identifier (ID), and a care provider context. The method also includes querying a patient data store for patient records based on the patient ID, care provider ID, and care provider context, and performing two or more different types of filtering operations of the returned results to generate a final reduced set of patient records. The method can also include transmitting, to the user device, a response comprising at least a subset of the final reduced set of patient records.

### BACKGROUND

Medical treatment is becoming increasingly complex. There are a number of different types of care providers, such as doctors, specialists, nurse practitioners, physician assistances, registered nurses, dieticians, social workers, and others that may be involved in the care of a single patient. Each of these practitioners generates patient care data in the form of notes, orders, medical records, medical imaging, tests, appointments, and so on, which are stored in electronic form in a centralized data repository. Thus, when each of the above-mentioned care providers is to provide care to a patient, the care provider should have sufficient information to provide the best care for the patient. However, sifting through structured and unstructured patient data is often difficult given the different sources of data, providers that generated data, storage locations of the data, etc.

Therefore, a technical challenge of how to transform the various forms of data from the different locations in a way that consolidates the data into a common storage form is created. Further technical challenges are created in how to find, access, transform, and provide relevant data to specific providers in a timely and summarized manner are also created. Additionally, in modern remote communications systems, how to then provide and present such data to the various providers that may be remotely distributed at one or more computing and/or telecommunications networks is further created.

### BRIEF DESCRIPTION OF THE DRAWINGS

The present disclosure will be understood more fully from the detailed description given below and from the accompanying drawings of various embodiments, which, however, should not be taken to limit the embodiments described and illustrated herein, but are for explanation and understanding only.

FIG. 1 is a block diagram of an exemplary system architecture for a care management platform providing secure real-time provider intelligence information.

FIG. 2 is a block diagram of one embodiment of another embodiment of an architecture of a care management platform, a user device, and one or more medical information provider system(s).

FIG. 3 is a block diagram of a process of a data extraction engine gathering, extracting, and storing patient data for use in providing secure real-time provider intelligence information.

FIG. 4 is a flow diagram of one embodiment of a process of a recommendation engine surfacing and transmitting relevant patient data in real-time to supply provider intelligence information.

FIG. 5 is a flow diagram of one embodiment of a process of a semantic search and question engine providing smart responses to provider queries for patient data in real-time.

FIG. 6 is a flow diagram of one embodiment of a process of a care management platform and a user device interacting during the providing of secure real-time provider intelligence information with machine learning retraining feedback.

FIG. 7 is a flow diagram of one embodiment of a process of a care management platform and a user device interacting during a text or natural language query to provide semantic search results as provider intelligence information.

FIG. 8 is one embodiment of a computer system that may be used to support the systems and operations discussed herein.

FIG. 9 is one embodiment of a mobile device that may be used to support the systems and operations discussed herein.

### DETAILED DESCRIPTION

In the following description, numerous details are set forth. It will be apparent, however, to one of ordinary skill in the art having the benefit of this disclosure, that the embodiments described herein may be practiced without these specific details. In some instances, well-known structures and devices are shown in block diagram form, rather than in detail, in order to avoid obscuring the embodiments described herein.

Some portions of the detailed description that follow are presented in terms of algorithms and symbolic representations of operations on data bits within a computer memory. These algorithmic descriptions and representations are the means used by those skilled in the data processing arts to most effectively convey the substance of their work to others skilled in the art. An algorithm is here, and generally, conceived to be a self-consistent sequence of steps leading to a desired result. The steps are those requiring physical manipulations of physical quantities. Usually, though not necessarily, these quantities take the form of electrical or magnetic signals capable of being stored, transferred, combined, compared, and otherwise manipulated. It has proven convenient at times, principally for reasons of common usage, to refer to these signals as bits, values, elements, symbols, characters, terms, numbers, or the like.

It should be borne in mind, however, that all of these and similar terms are to be associated with the appropriate physical quantities and are merely convenient labels applied to these quantities. Unless specifically stated otherwise as apparent from the following discussion, it is appreciated that throughout the description, discussions utilizing terms such as “receiving”, “initiating”, “querying”, “applying”, “selecting”, “generating”, “transmitting”, “adding”, “retraining”, “executing”, “extracting”, “converting”, “determining”, or the like, refer to the actions and processes of a computer system, or similar electronic computing device, that manipulates and transforms data represented as physical (e.g., electronic) quantities within the computer system's registers and memories into other data similarly represented as physical quantities within the computer system memories or registers or other such information storage, transmission or display devices.

The embodiments discussed herein may also relate to an apparatus for performing the operations herein. This apparatus may be specially constructed for the required purposes, or it may comprise a general-purpose computer selectively activated or reconfigured by a computer program stored in the computer. Such a computer program may be stored in a computer readable storage medium, such as, but not limited to, any type of disk including floppy disks, optical disks, CD-ROMs, and magnetic-optical disks, read-only memories (ROMs), random access memories (RAMs), EPROMs, EEPROMs, magnetic or optical cards, or any type of media suitable for storing electronic instructions.

The algorithms and displays presented herein are not inherently related to any particular computer or other apparatus. Various general-purpose systems may be used with programs in accordance with the teachings herein, or it may prove convenient to construct a more specialized apparatus to perform the required method steps. The required structure for a variety of these systems will appear from the description below. In addition, the embodiments discussed herein are not described with reference to any particular programming language. It will be appreciated that a variety of programming languages may be used to implement the teachings as described herein.

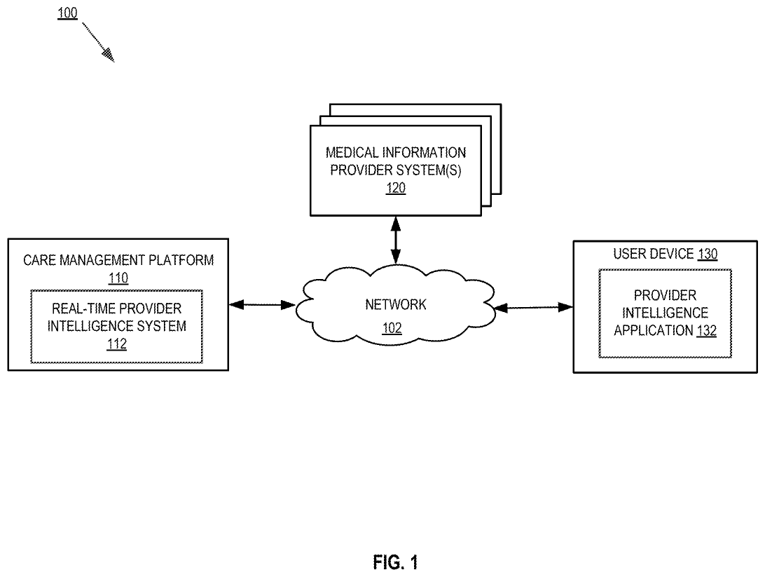

FIG. 1 is a block diagram of an exemplary system architecture 100 for a care management platform providing secure real-time provider intelligence information. In one embodiment, the system 100 includes a care management platform 110, one or more medical information provider system(s) 120, and user device 130. As will be discussed in greater detail below, the user device 130 is associated with a care provider, such as a doctor, specialist, nurse practitioner, dietician, social worker, etc. providing care to a patient. Furthermore, there may be any number of user devices associated with the same and/or different care providers that simultaneously utilize the real-time provider intelligence services of the care management platform 110. However, to avoid obscuring the techniques discussed herein, only a single user device 130 is shown.

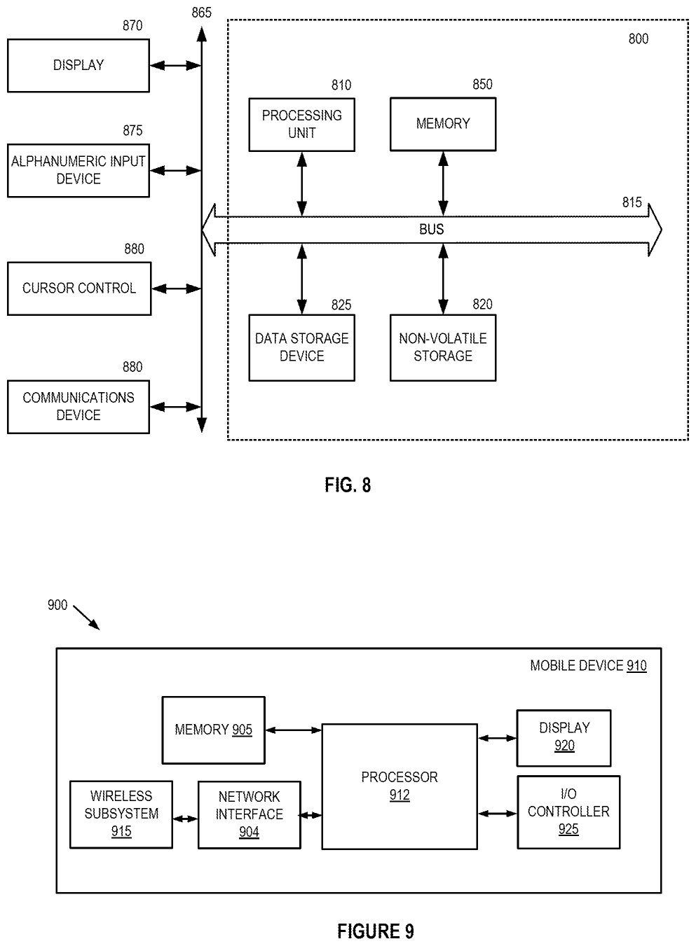

In one embodiment, user device 130 may be mobile computing device, such as a smartphone, tablet computer, smartwatch, etc., such as is illustrated and described with respect to FIG. 9. User device 130 may also be a computer system, such as a desktop computer system, laptop computer system, terminal, etc., such as is illustrated and described with respect to FIG. 8. The care management platform 110 may also be one or more computing devices, such as one or more server computer systems, desktop computer systems, etc., such as is illustrated and described with respect to FIG. 8.

The care management platform 110, one or more medical information provider system(s) 120, and user device 130 may be coupled to a network 102 and communicate with one another using any of the standard protocols for the exchange of information, including secure communication protocols such as hypertext transfer protocol secure (HTTPS) and/or transport layer security (TLS), secure socket layer (SSL), or other secure protocols for the exchange of information over a network. In one embodiment, one or more of the care management platform 110, one or more medical information provider system(s) 120, and user device 130 may run on one Local Area Network (LAN) and may be incorporated into the same physical or logical system, or different physical or logical systems. Alternatively, the care management platform 110, one or more medical information provider system(s) 120, and user device 130 may reside on different LANs, wide area networks, cellular telephone networks, etc. that may be coupled together via the Internet but separated by firewalls, routers, and/or other network devices. In one embodiment, care management platform 110 may reside on a single server, or be distributed among different servers, coupled to other devices via a public network (e.g., the Internet) or a private network (e.g., LAN). It should be noted that various other network configurations can be used including, for example, hosted configurations, distributed configurations, centralized configurations, etc.

In one embodiment, care management platform 110 provides real-time provider intelligence information to user device 130. The real-time provider intelligence information, as discussed herein, is patient related data that is relevant to a particular patient, relevant to provider type of the current provider, and relevant to a domain in which care is being provided. For example, a doctor (e.g., provider type as doctor) providing kidney care services (e.g., domain of kidney care) to a particular patient would find specific patient information relevant for providing optimal care that may not be relevant to a dietitian or social work providing care to the same patient. This is because the provider type, and thus the care services and relevant information are likely different among the doctor, dietician, social work, as well as other care provider types. Therefore, in embodiments, as will be discussed in greater detail below, real-time provider intelligence system 112 provides care providers (e.g., a user of user device 130) access to real-time summarized data produced by a machine learning (ML) powered recommendation engine and served via application programming interface (API) based messaging, enables searching of patient data using one or more search processes, such as text search and/or smart semantic search, also served via API messaging, and enables end user to provide feedback to the recommendation, search, and question answering engines through contact with user interface elements in a graphical user interface (GUI) displayed by the provider intelligence application 132. Furthermore, real-time provider intelligence system 112 of the care management platform is able to ingest, transform, extract, and annotate patient data received in various forms such as structured data (e.g., forms, database records, etc.) and unstructured data (e.g., document images, textual passages from patient records, care provider visit notes, etc.). These techniques, as disused herein, solve the technical challenges of how to transform the various forms of data from the different locations in a way that consolidates the data into a common storage form is created, how to find, access, transform, and provide relevant data to specific providers in a timely and summarized manner are also created, how to consistently improve the underlying ML process that enable the recommendations and search services using real-time provider feedback of the very providers of the various types for which the ML models are trained to recognize, and how to then provide and present such data to the various providers that may be remotely distributed at one or more computing and/or telecommunications networks is further created.

As discussed herein, care management platform 110 is enabled to provide real-time provider intelligence in a domain of care, such as kidney care, heart care, weight management, joint health, skin cancer care, as well as various other domains of patient care. In some embodiments, care management platform 110 is enabled to provide real-time provider intelligence in a number of different domains of medical care. To avoid obscuring the embodiments of the present invention, the discussion below and the examples herein will focus on the medical care domain of kidney care.

In order to provide real-time care provider intelligence information, real-time provider intelligence system 112 continuously gathers relevant patient records and documentation from the one or more medical information provider system(s) 120. The access to patient data is predicated on patient consent to allow care management platform 110 access to patient data, and transmission of such information occurs over secure communications channels, such HTTPS for example. Furthermore, the medical information provider system(s) 120 may include one or more of server computer systems having electronic records stored therein, including patient notes, charts, tests, imaging, etc., communication systems (e.g., facsimile machines) which push scans, PDFs, or other document images to real-time provider intelligence system 112, or other information storage and/or provisioning systems. Furthermore, each of the one or more medical information provider system(s) 120 may be associated with the same institution (e.g., hospital, clinic, office, etc.) providing care to patients, or may be associated with other institutions. Thus, in embodiments, real-time provider intelligence system 112 may pull patient data from the one or more medical information provider system(s) 120 and/or receive pushed patient data from the one or more medical information provider system(s) 120. This process pull-based data gathering is performed periodically, such as hourly, daily, weekly, etc., and receiving pushed based patient data is performed in response to data transmissions.

In response to the data gathering activities, real-time provider intelligence system 112 then ingests and extracts key information from the patient data records. In embodiments, real-time provider intelligence system 112 scans each obtained medical document for information relevant to the provision of care management services, such as kidney care. This information includes patient information, provider information, outpatient appointment and inpatient admission dates, and medical concepts including medications, anatomical sites, medical procedures, symptoms, diseases, and so on. The extracted information is then added as metadata to the obtained medical document. Furthermore, where applicable, the extracted information is further mapped to Concept Unique Identifiers (CUI) from the Unified Medical Language System (UMLS) to uniquely identify extracted information using the formalized CUI from the UMLS, and metadata updated to reflect the mapping including adding determined CUIs to associated extracted information. Additional metadata is also retained for the document, such as the location of the text in the document that has been extracted, a preferred text for a specific medical concept (e.g., as identified by a mapped CUI), the type of medical concepts associated with a document, etc.

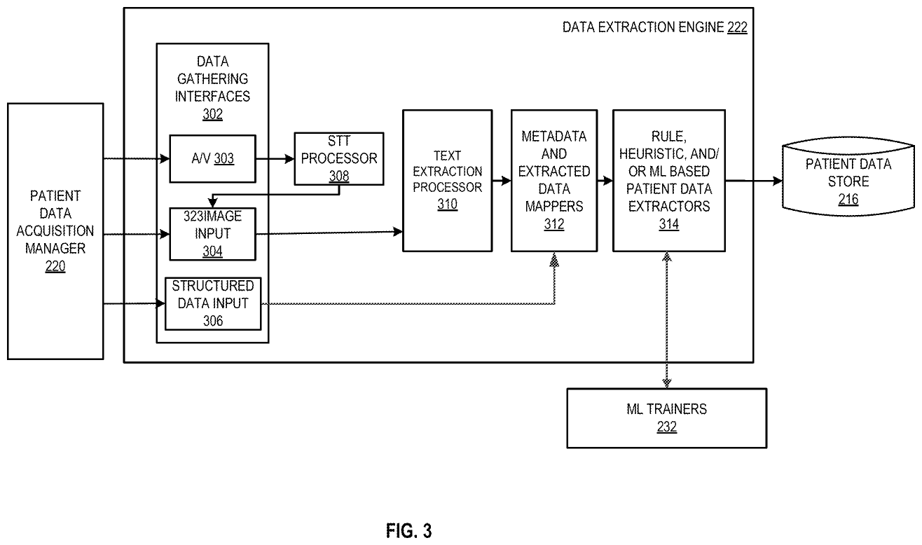

The extracted data is indexed and saved in a distributed document-based storage system, for example an inverted index distributed storage and search system such as a system based on Lucene, to enable flexible storage that is scalable based on volume of data, load, regional access capabilities, etc. Furthermore, the data ingestion and information extraction process to obtain, transform, and store the original medical records to a format suitable for the intelligence processes discussed herein, and as discussed more fully in FIG. 3, may be parallelized by executing multiple instances of the data gathering and data extraction processes based on current computing loads. The data gathered and information extracted for multiple patients, including original document images, extracted information (e.g., patient information, provider information, medical concepts, mappings to CUIs, locations of text within original documents, etc.), patient identifiers, etc. therefore forms as corpus of patient information upon which the processes discussed herein operate.

Real-time provider intelligence system 112 then accesses the corpus of patient information to provide care related information to provider intelligence application 132 executed on user device 130. As discussed herein, user device 130 is associated with a care provider (e.g., doctor, RN, NP, dietician, specialist, social worker, etc.). In embodiments, provider intelligence application is an API based application that transmits a request for patient information to an API endpoint of real-time provider intelligence system 112. The request is transmitted via network 102 and uses secure protocols for the exchange of the request and receipt of the response, such as HTTPS. The request can include one or more of a patient identifier, a care provider identifier, a care provider context (e.g., a type of the provider—doctor, RN, NP, specialist, etc.), optionally a care domain (e.g., kidney care, heart care, lung care, etc.), a combination of the aforementioned information, as well as one or more additional identifiers, context information, care domain information, etc. In some embodiments, real-time provider intelligence system 112 operates in a single care domain and thus the care domain need not be specified in the patient information request.

The real-time provider intelligence system 112, as discussed more fully below, accesses all records from the corpus of processed patient data associated with the identified patient. Then real-time provider intelligence system 112 performs a series of filtering and sorting of the previously extracted data based on the care domain and provider context to quickly find the most relevant and valuable patient data for providing care management services. The real-time provider intelligence system 112 transmits, via the API endpoint, the filtered and sorted patient data output via API based messaging back to the provider intelligence application 132 for rendering within a GUI of the provider intelligence application.

The GUI of the provider intelligence application 132 further enables care provider access to search services of the real-time provider intelligence system 112, as well as ML retraining feedback mechanisms.

In embodiments, the GUI of the provider intelligence application 132 displays one or more search interfaces for receipt of search request of a user of user device 130. A first type of search request includes a keyword-based search query received from a user of user device 130, and transmitted via API messaging to the real-time provider intelligence system 112. However, the provider intelligence application will add patient identifier(s), provider identifier(s), and provider context to the search keywords to ensure a more relevant search is performed. A search engine, such as an inverted index based search engine then performs data retrieval on the corpus of indexed patient information using the keywords, patient ID, provider ID, and provider context. In embodiments, the search finds phrases in the patient's medical records that are good result candidates, scores them based on relevancy using the provider context, sorts them, and uses ML methods to select the best results, such as decision tree based selection techniques, neural network based selection techniques, another ML based selection technique, as well as an ensemble of multiple ML based selection techniques. As discussed herein, the ML engines that select best results are pretrained an then retrained based on hand-labeled training data and provider feedback. These results are then returned as an API response to the API request and rendered in the GUI of the provider intelligence application 132. Again, because these searches involve the transfer of sensitive patient information, the transmission of the search request and responses use secure communications protocol.

In embodiments, a second type of search provided by the provider intelligence application 132 is a semantic and/or natural language question-based search. As opposed to keyword-based searches, the semantic and/or natural language question based search enables care providers to input more natural or “plain text” question (e.g., “When was the last blood draw that measured X in Patient Y?”), and receive semantically relevant answers from the patient's medical records (e.g., the corpus of extracted patient information). The provider intelligence application 132 responsive to a question type search input, transmits the search string, patient identifiers, provider identifiers, and provider context as an API based message to the endpoint of real-time provider intelligence system 112. Whereas the first type of search discussed above functions by mostly matching words, the second type of search extracts semantic meaning form the query string (e.g., by extracting various entities from the string, determining intents of those entities, and determining likely semantic meaning using, for example, a text vectorization process). That is, the second type of search finds semantically similar phrases or answers to the question in medical records matching a particular patient. In embodiments, real-time provider intelligence system does this matching by converting both the question string and all potential answers in the medical records to mathematical structures that contain semantic meaning. For example, the question string and answers are converted to a vector or matrix that indicates the presence or absence of specific features, concepts, medial identifiers, etc. Then, in some embodiments, a proximity measurement is computed, for example a Euclidean or cosine distance, with a subset of answers proximate to the question string being selected and returned to the provider intelligence application. In other embodiments, a predictive based technique, such as a generative ML model based technique, predicts an answer or subset of answers based on the words and/or sequence of words in the query string and the medical records as a source of the words in the predictive answer(s). The subset of answers are returned via API messaging and secure communications to populate the GUI of the provider intelligence application 132.

In some embodiments, the first type of search including a keyword search may also utilize the semantic intelligence search process. That is the keywords may form the search string, analyzed and converted to a mathematical construct, and then judged for proximity to potential answers, as discussed herein. Thus, in this embodiment, even the text/keyword based search returns intelligent answers.

In embodiments, several of the processes enabling real-time retrieval and consolidation of recommendations and searches involve using ML based processes. Therefore, in embodiments, provider intelligence application 132 provides one or more GUI elements to provide care provider feedback on recommendation and/or search results. For example, a care provider can flag certain recommendations and/or search results as being particularly good for the provider context, with the recommendation, original recommendation query, and provider feedback information added to a set of ML retraining data of the real-time provider intelligence system 112. As another example, a care provider can flag other recommendations and/or search results as not relevant, good, etc., and identify the area of failure (e.g., wrong provider context, old data, irrelevant to the care domain, irrelevant section of a medical document, etc.). Again, this can be added to a set of ML retraining data. Then, periodically, the various ML models (e.g., tree based, neural network based, generative, etc.) can be retrained at least in part on the care provider feedback. As a result, the ML models can adapt and improve to reflect the real world and real-time needs of the various types of care providers and the recommendations and query results provided thereto.

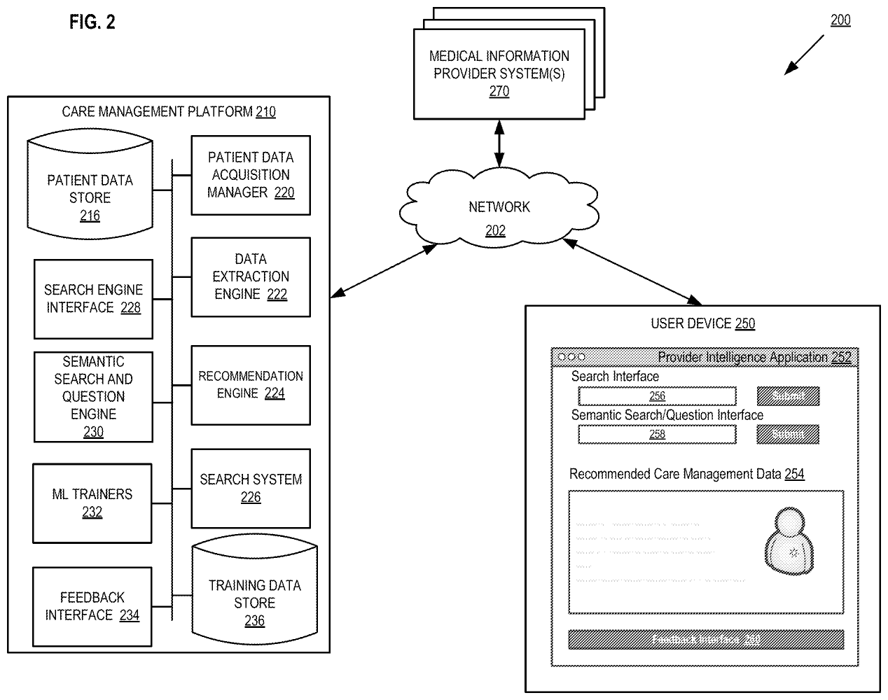

FIG. 2 is a block diagram of one embodiment of another embodiment of an architecture 200 of a care management platform 210, a user device 250, and one or more medical information provider system(s) 270. Care management platform 210 and user device 250 provide additional details for the corresponding devices/systems discussed above in FIG. 1.

In one embodiment, care management platform 210 includes a patient data acquisition manager 220, a data extraction engine 222, a recommendation engine 224, a search system 226, a patient data store 216, a search engine interface 228, a semantic search and question engine 230, ML trainers 232, a feedback interface 234, and a training data store 236. Furthermore, in one embodiment, user device is configured to execute a provider intelligence application 252 having a GUI that includes at least one or more search interfaces 256 and/or 258, recommended care management data 254, and a feedback interface 260.

Care management platform 210, as discussed herein, ingests various forms of patient medical records from the one or more medical information provider system(s) 270. Patient data acquisition manager 220 is an interface that is responsible for receiving patient records data pushed to the care management platform 210, such as a fax or email interface for receiving patient record document images, PDFs, or other document images. Furthermore, patient data acquisition manager 220 is also configured to periodically query medical information provider system(s) 270 for new patient records, such as new notes, appointments, test results, medical images, etc. generated at the medical information provider system(s) 270 since a last query. Furthermore, these queries can specify the patient identifier for which patient data store 216 maintains records. Furthermore, the pushed and queried data can be used to obtain various types of medical records for patients, in both structured (e.g., forms, XML documents, spreadsheets, database records, etc.) as well as unstructured data (e.g., fields in forms corresponding to doctor or nurse notes, images, etc.). This information is then passed to the data extraction engine 222.

Data extraction engine 222 is responsible for processing the patient records to transform received documents, extract relevant information, and supplement the patient records with CUIs, document locations, etc. for the extracted relevant information. One embodiment of a data extraction engine 222 is illustrated in FIG. 3.

FIG. 3 illustrates a process of a data extraction engine gathering, extracting, and storing patient data for use in providing secure real-time provider intelligence information. The process is performed by processing logic that may comprise hardware (circuitry, dedicated logic, etc.), software (such as is run on a general-purpose computer system or a dedicated machine), firmware, or a combination.

With reference to FIG. 3, the data gathering interfaces 302 of the data extraction engine 222 receiving one or more structured and/or unstructured patient records from the patient data acquisition manager 220. The records may be audio (e.g., recordings) or audiovisual (e.g., video), which are revived by A/V input 303 and provided to a speech-to-text processor 308 to extract textual information from any audio contained within the AV record (e.g., spoken provider content or spoken patient content). The extracted textual information is then added to the image (if any) and forwarded to image input 304, which forwards the image and textual information to text extraction processor 310. In some embodiments, a medical image may not include audio content (e.g., x-rays, MRIs, or other medical imaging), and is therefore forwarded directly from the image input 304 to text extraction processor 310.

Text extraction processor 310 performs a set of one or more image processing operations, including image enhancement, contrast adjustment, skew correction, etc. to format a received image associated with a patient medical record. Furthermore, segmentation of text within an image may then be performed so that text extraction can also be performed to extract textual content form the images. For example, this can include extracting text from forms, extracting text from medical imaging, and so on. The extracted textual content is then forwarded to mappers 312.

In some embodiments, structured patient data records (e.g., forms, XML documents, etc.) may be received by structured data input 306, which passes the text from the structured data record directly to mapper 312.

Metadata and extracted data mappers 312 receive the textual content, and supplement the text extracted from the original patient record with CUI assignments. In embodiments, mappers 312 will map one or more UMLS CUIs to text strings and/or passages that match terms of the UMLS CUIs.

Rule, heuristic, and/or ML based patient data extractors 314 are a set of process operations that perform one or more extraction operations to determine and filter relevant patient data from all identified textual content. That is, a form may specify non-relevant content (e.g., form fields, name of institution, boiler plate language, etc.), and rule, heuristic, and/or ML based patient data extractors 314 is configured to recognize relevant patient information (e.g., patient data relevant to a care domain, such as patient data relevant to kidney care) from among all the textual data. Therefore, in embodiments, a set of extractors are executed progressively including a rule and/or heuristic extractor and then an ML based patient data extractor. The rule and/or heuristic extractor of the rule, heuristic, and/or ML based patient data extractors 314 filters or excludes content based on preset rules. For example, terms, locations on document, etc. can form rules that filter out content known to be irrelevant to patient care. Then, the remaining textual content is provided to an ML patient data extractor.

The ML patient data extractor is pretrained and retrained by ML trainers 232 to recognize highly relevant textual content for a care domain and/or provider context. For example, the ML patient data extractor may be a tree, neural network, or other form of ML based model, as well as ensembles of one or more different ML models, that are initially trained on hand-labeled training data that specified relevant and non-relevant patient data text. In embodiments, ML trainers 232 perform ML patient data extraction training using ML features that include the extracted text from the medical record, any extracted metadata, information about the patient, information about the care provider, CUIs assigned to text, and information about care provider context. Once trained to a sufficient level of accuracy, the ML model(s) can progress to production phase where the one or more ML models perform extraction by scoring textual content not already filtered, and from which content satisfying a relevancy score threshold is identified (and content not satisfying the relevancy score threshold is excluded). Furthermore, ML trainers 232, as discussed herein may use user feedback received from feedback interface 260, which provide user scoring of the real-time recommendations as retraining data for storage in store 236. The retaining data enables ML trainers 232 to optimize the text relevance scoring by periodically retraining the ML model(s) using feedback on real-time scoring by the care providers for which relevancy is being judged.

Once the extractors 314 finish their filtering and identification of the relevant care information in a patient record, which is modified, annotated, added with metadata, etc., the extracted information as well as the original patient record, image, document, AV recording, etc. is added to patient data store 216.

Returning to FIG. 2, in embodiments, extractors 314 provide the extracted information and the patient record to search system 226 for storage in patient data store 216. As discussed above, search system 226 performs document-based storage. and indexing. Furthermore, in some embodiments, search system 226 is a document storage and search system that stores the received data in a distributed manner, performs indexing prior to storage, and stores routing information and search indexes for access to the distributed data stores of patient data store 216. In some embodiments, search system 226 is an inverted index based document storage and search system.

In embodiments, recommendation engine 224 includes an API interface (e.g., endpoint) for receiving recommendation request messages from provider intelligence application 252. In an embodiment, provider intelligence application 252 generates a request, for example in response to a user request, in response to a calendar event, in response to an appointment with a patient, at startup of the application, etc., for a set of relevant patient information for a provider context associated with the user of user device 250. In an embodiment, provider intelligence application 252 is integrated with an API and/or software development kit (SDK) of the care management platform 210 for generating an API request including a patient identifier to identify the particular patient to which a request is directed, a provider identifier to identify the care provider making the request, a care provider context that identifies the role or type of care provider (e.g., doctor, specialist, RN, NP, social work, etc.), and optionally a care provider domain (e.g., kidney care, lung care, heart care, etc.). In some embodiments, the domain is inherent in the system and the system only applies to the specific domain. The API message, requesting patient information that is relevant to the care provider context is then transmitted from application 252 to the endpoint exposing recommendation engine 224.

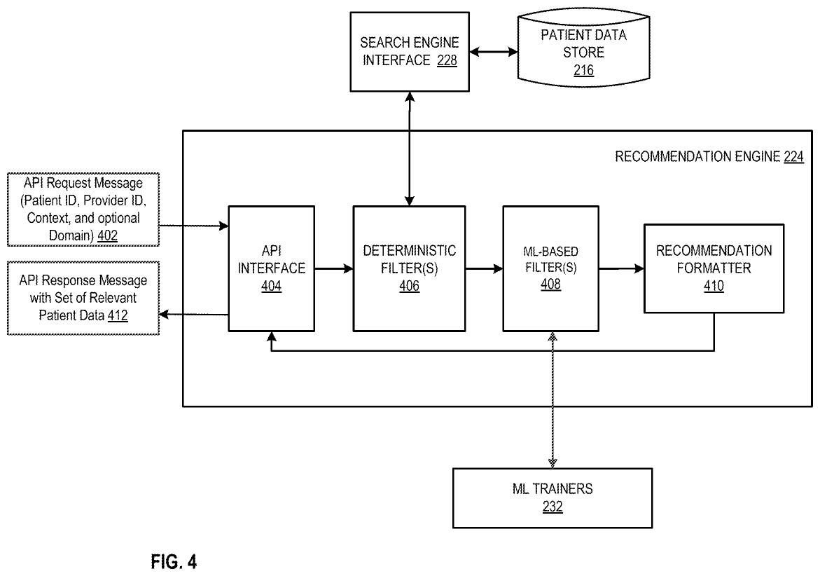

FIG. 4 illustrates one embodiment of a process of a recommendation engine surfacing and transmitting relevant patient data in real-time to supply provider intelligence information. The process is performed by processing logic that may comprise hardware (circuitry, dedicated logic, etc.), software (such as is run on a general purpose computer system or a dedicated machine), firmware, or a combination.

With reference to FIG. 4, recommendation engine 224 includes the API interface 404 acting as an API endpoint for receiving the API request message 402. The message 402, which can include the patient ID, care provider ID, care provider context, and optional domain, is forwarded to deterministic filters 406. In embodiments, the message 402 can include more or less information than that described above, as well as other identifiers, context information, domain information, etc. that are relevant to the recommendations being generated by recommendation engine 224. Deterministic filters 406 are responsible for initiating one or more queries, using the search engine interface 228, for patient data records associated with the patient ID in message 402. This can include search engine interface 228 directly looking up records in patient data store 216, or issuing search requests to the search system 226. In either embodiment, a plurality of potentially relevant patient records and/or extracted data is returned to deterministic filters 406.

Deterministic filters 406 then apply a first filtering of the potentially relevant patient records by applying one or more rule or heuristic based filters. The heuristic-based filters seek to exclude non-relevant patient records or information. For example, the filters can include filtering patient data associated with a care provider context that isn't the current care provider context, filter data older than a certain date, filter data not having a determined relevancy, filtering data with CUIs only associated with non-care provider contexts, filtering system-generated language commonly found in unstructured medical note data, as well as other heuristic or rule-based filters. The filtering of deterministic filters 406 performs a first data reduction by eliminating non-relevant patient data. Experimental results have shown that a significant number of patient records and/or extracted data can be filtered out at this stage, which enables improved accuracy and improved computational efficiency in further searching the remaining records.

The remaining patient records and/or extracted data are then provided to ML-based filter(s) 408. As discussed above, the ML-based filter(s) 408 may filter or select patient records or portions of patient records using one or more neural network based ML model(s), tree-based ML model(s), generative and predictive model(s), as well as other ML model(s) and/or ensemble of models, and thus are trained by ML trainers 232 prior to production. In embodiments, as discussed herein, initial training data includes hand labeled data related in patent records, extracted information, care provider context, and other metadata relevant to patient information. This is used to initially train the one or more ML-based filter(s) 408 to score relevancy of patient information for a given care provider context. Furthermore, once in production, ML trainers 232 may access retraining data in a retraining data store (e.g., data store 236), including at least a portion of care provider feedback as the relevancy and/or lack thereof of specific recommendations. Thus, ML-based filter(s) are continually being optimized based on real-time and real world care provider feedback.

After ML-based filter(s) 408 perform relevancy scoring for a given patient's information for a given provider in a given context, a subset of patient information have the top scores are selected and provided to recommendation formatter 410. Recommendation formatter 410 transforms the recommendations into a data format suitable for API response message 412. Furthermore, recommendation formatter may supplement data records with links to an original medical document or record associated with identified relevant patient information, which links the recommendation to the specific patient record from which the recommendation was generated.

The formatted recommendations are then provided to API interface 404, which packages the recommendations in API response message 412. The message 412 is then transmitted back to provider intelligence application 252 for populating the recommended care management data UI 254.

Returning to FIG. 2, responsive to the display of the recommendations in application 252, user interface may receive one or more queries from a user of user device 250. In one embodiment, the query may be a keyword-based query entered into search interface 256 or a semantic search/question entered into interface 258. In some embodiments only one of the interfaces is rendered by a GUI. Furthermore, a user may request the search functions within the application 242, such as by selecting a menu option, icon, or other user interface element (not shown).

The keyword based search received at search interface 256 transmits the keywords to search engine interface 228, for example as an API based message, which is passed to search system 236 for performing a keyword based search on data store 216. Search system 226 or search engine interface 228 may then return results to populate a Ul element of application 252, for example, element 254.

In some embodiments, only the semantic search/question interface is rendered within application 252. That is, all text and natural language based searches are treated as semantic queries in which a context and/or meaning of the query informs the obtaining of query results. The search is transmitted, for example as an API based message to semantic search and question engine 230.

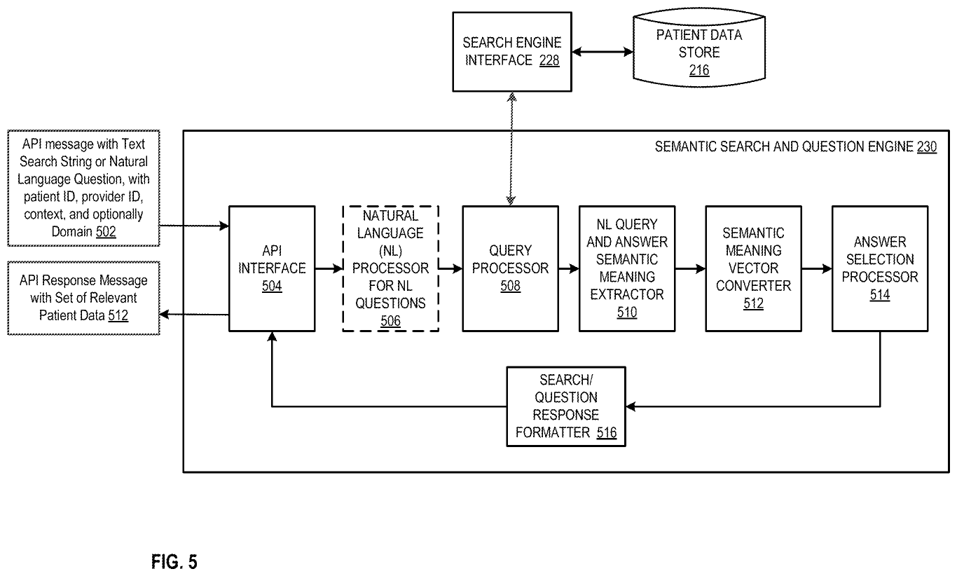

FIG. 5 illustrates a process of a semantic search and question engine 230 providing smart responses to provider queries for patient data in real-time. The process is performed by processing logic that may comprise hardware (circuitry, dedicated logic, etc.), software (such as is run on a general purpose computer system or a dedicated machine), firmware, or a combination.

With reference to FIG. 5, API interface 504 receives the text search string or natural language question in API message 502. To aid in the semantic analysis and patient information retrieval, the API message 502 also includes the patient ID, provider ID, provider context, and optionally a care provider domain. API interfaces passes the API message contents to NL processor 506.

In embodiments, NL processor 506 is optional for natural language questions. As discussed herein, those are not keyword based searches and are instead searches typically forming complete and conversation type questions (e.g., “When was the patients last MRI?”, “Where can I find a blood count in the last 5 blood tests?”, etc.). NL processor 506 executes one or more NL processing operations to convert the search question string into a search that can be executed by search engine 228.

Query processor 508 receives the keyword or NL processed query terms and executes a search against patient data store 216 using search engine interface, as discussed above. A plurality of results are returned by search engine interface 228, and forwarded to NL query and answer semantic meaning extractor 510.

NL query and answer semantic meaning extractor 510 receives the results and then determines semantic meanings associated with the original query and returned answers. For example, NL query and answer semantic meaning extractor 510 performs operations, such as entity identification in a search string, intent determination, etc. to obtain semantic meaning indicators from terms/features of the search string and/or returned answers.

Once the relevant terms/features are identified in the strings of the original query and the returned answers, semantic meaning vector converter 512 converts the strings and/or identified terms/features into mathematical constructs. In one embodiment, semantic meaning converter 512 converts the strings, such as the entire strings, just the identified terms/features, or a combination to a matrix where each row vector of a matrix maps to a word of the string, such that each vector contains contextual information associated with that specific word. Then, semantic meaning converter 512 then flattens the matrices to vectors. Thus, the semantic meaning vector converter 512 is able generate a vector representation of a search string.

Answer selection processor 514 then selects and/or generates one or more answers using the vector representation of the search string. In one embodiment, proximity processing is performed to determine how close a search vector is to potential results vectors. As discussed herein, the results vectors may be generated from patient medical records and/or portions of patient medical records by converting those records/portions into mathematical constructs, such as how the search string is converted to a vector representation. In an embodiment, proximity processing performs a distance calculation to determine how far or close a search vector is from each results vector. In another embodiment, the search vector and/or words making up the search vector may be used by a predicative technique (e.g., a trained generative ML model) that predicts the words or answers based on patient records on one or more potential results. A subset of the results which are determined to be best matches to the search vector are returned to search/question response formatter 516.

As discussed above, the formatter 516 transforms the selected subset of search results (e.g., the semantically closest search results) into a form suitable for an API based response to the application 252. API interface 504 receives the formatted subset of search results and transmits response message 512 with the set of relevant patient data.

Returning to FIG. 2, provider intelligence application 252 receives the results and renders them on the GUI of application 252, for example, in data field 254.

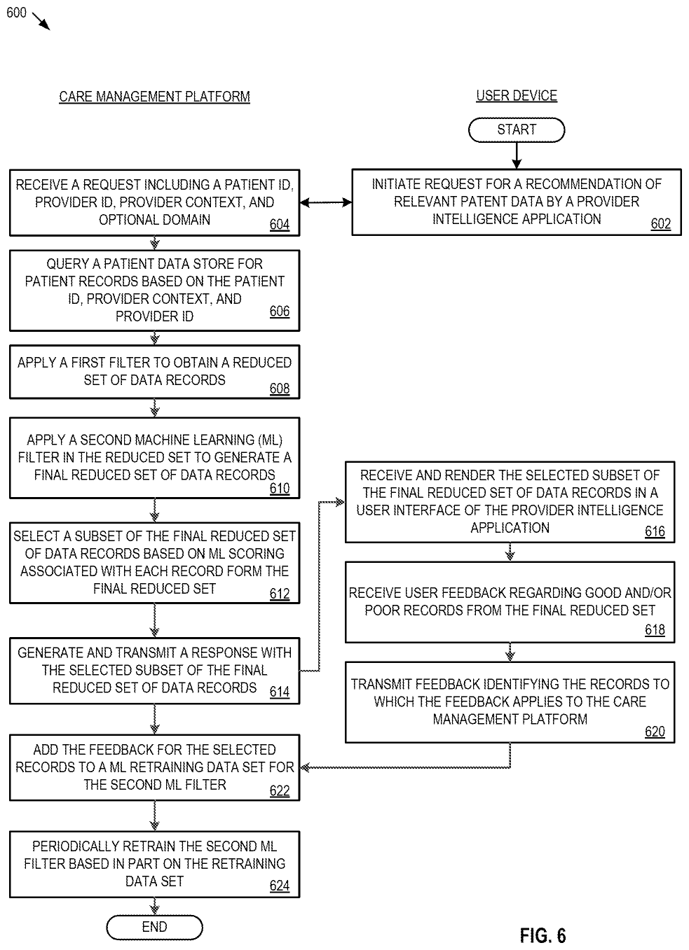

FIG. 6 is a flow diagram of one embodiment of a process 600 of a care management platform and a user device interacting during the providing of secure real-time provider intelligence information with machine learning retraining feedback. The process 600 is performed by processing logic that may comprise hardware (circuitry, dedicated logic, etc.), software (such as is run on a general purpose computer system or a dedicated machine), firmware, or a combination. In one embodiment, the process is performed by a care management platform (e.g., platform 110 or 210) and a user device (e.g., user device 130 or 250).

Referring to FIG. 6, processing logic of the user device begins by initiating a request for a recommendation of relevant patent data by a provider intelligence application (processing block 602). The request is an API message request sent to an API endpoint of a care management platform, and the message payload includes a patient identifier, a care provider identifier, a care provider context, and optionally a care domain. However, as discussed herein, the care domain may be inherent in the request.

Processing logic of the care management platform receives the request including the patient ID, provider ID, provider context, and optional domain (processing block 604). With this information, processing logic then queries a patient data store for patient records based on the patient ID, provider context, and provider ID (processing block 606).

Using the query results and to reduce the total results to relevant results, processing logic further applies a first filter to obtain a reduced set of data records (processing block 608). The first filter, in embodiments, is one or more of a rule based filter or a heuristic based filter that exclude from the query results any query matching one or more of the rules and/or heuristics.

Processing logic then applies a second machine learning (ML) filter on the reduced set to generate a final reduced set of data records (processing block 610). In one embodiment, as discussed herein, the second ML filter is trained to determine a relevance of a returned result (e.g., piece of extracted patient information, medical record, medical image, etc.) based on one or more mapped identifiers. That is, during data extraction, one or more UMLS CUIs are assigned to records, patient information, etc. Thus, the second ML filter is trained to score a relevance of patient information based on a combination of one or more of those CUIs and a provider context.

Processing logic uses the scoring to select a subset of the final reduced set of data records based on ML scoring associated with each record form the final reduced set (processing block 612). In embodiment, the subset comprises the top N scoring results. In another embodiment, all results exceeding a predetermined threshold may be selected for the subset of the final reduced set of data records.

Processing logic then generate and transmits a response with the selected subset of the final reduced set of data records (processing block 614), and processing logic of the user device receives and renders the selected subset of the final reduced set of data records in a user interface of the provider intelligence application (processing block 616).

In some embodiments, and for some entries in the subset of the final reduced set of data records rendered in the user interface, a user may determine to provide feedback on results. For example, a user may select a user interface element to initiate this feedback process. Processing logic then optionally receives user feedback regarding good and/or poor records from the final reduced set (processing block 618), and transmits feedback identifying the records to which the feedback applies to the care management platform (processing block 620).

Processing logic of the care management platform adds the feedback for the selected records to a ML retraining data set for the second ML filter (processing block 622). Processing logic then periodically retrains the second ML filter based in part on the retraining data set (processing block 624). The feedback is a response of the actual care providers to the recommended patient data records. Thus, their feedback (positive and negative) enables the retraining process to optimize the recommendations based on real world scenarios to reflect the actual information that will be most useful in each care provider context.

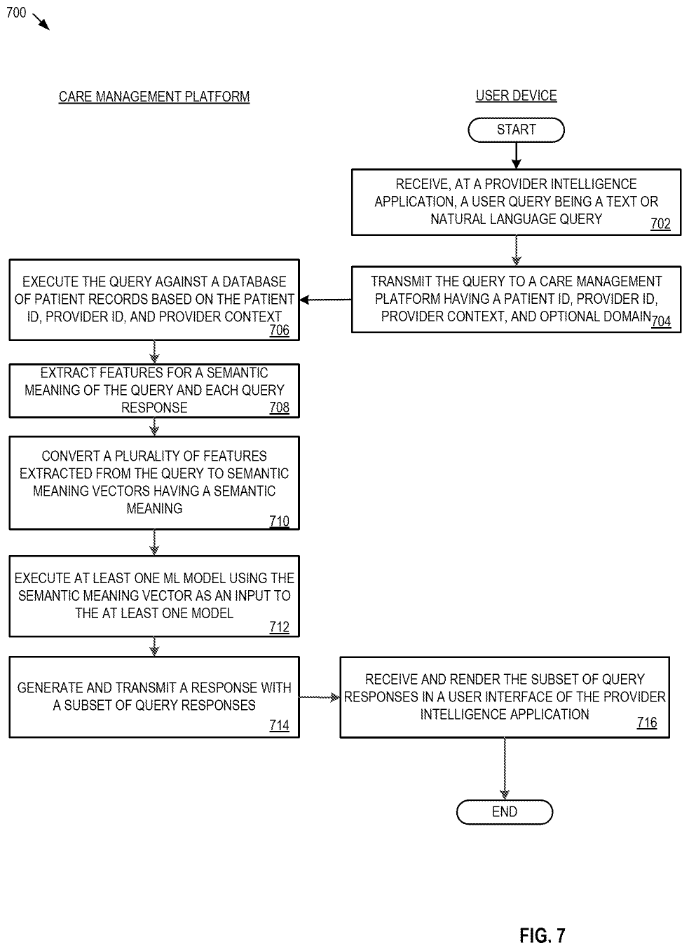

FIG. 7 is a flow diagram of one embodiment of a process 700 of a care management platform and a user device interacting during a text or natural language query to provide semantic search results as provider intelligence information. The process 700 is performed by processing logic that may comprise hardware (circuitry, dedicated logic, etc.), software (such as is run on a general purpose computer system or a dedicated machine), firmware, or a combination. In one embodiment, the process is performed by a care management platform (e.g., platform 110 or 210) and a user device (e.g., user device 130 or 250).

Referring to FIG. 7, processing logic of the user device begins by receiving, at a provider intelligence application, a user query being a text or natural language query (processing block 702). The query, as discussed herein, can include keywords (e.g., “kidney dialysis x-ray”) or a conversational query (e.g., “Has patient X had an x-ray of their kidneys since their last round of dialysis?”). Processing logic transmits the query to a care management platform having a patient ID, provider ID, provider context, and optional domain (processing block 704). As discussed herein the messaging is API based messaging using secure communication channels and/or protocols.

Processing logic of the care management platform, responsive to receipt of the query, executes the query against a database of patient records based on the patient ID, provider ID, and provider context (processing block 706). Processing logic then extracts feature for a semantic meaning of the query and each query response (processing block 708). As discussed herein, one or more NLP and semantic meaning processes are performed on the query itself and the returned responses to identify key terms/features relevant to determination of a semantic meaning, such as medical terms, identifiers, verbs, CUIs, etc.

Processing logic converts a plurality of features extracted from the query and each query response to semantic meaning vectors having a semantic meaning (processing block 710). As indicated above, these vectors are low-dimensional representations generated form the strings of the query and answers that can be used to infer the presence or absence of certain medical concepts or other important features. The values may be numerical quantities indicative of a contribution to relevance (e.g., feature X has a value of 2, whereas feature Y has a value of 0.6).

Processing logic then executes at least one ML model using the semantic meaning vector as an input to the at least one model (processing block 712). This model then determines a relevancy of the vector to possible search results. In an embodiment, the model determines a Euclidean distance, but other forms of distance measurements including cosine distance can be used, to determine a distance between the input vector and potential answers. In another embodiment, the input vector is input to a generative ML model that predicts the words of one or more responses. Processing logic then generates and transmit a response with a subset of query responses to the user device (processing block 714).

Processing logic of the user device receives and renders the subset of query responses in a user interface of the provider intelligence application (processing block 716).

FIG. 8 is one embodiment of a computer system that may be used to support the systems and operations discussed herein. For example, the computer system illustrated in FIG. 6 may be used by the care management platform, the medical information provider systems, as well as the user device. It will be apparent to those of ordinary skill in the art, however that other alternative systems of various system architectures may also be used.

The computer system illustrated in FIG. 8 includes a bus or other internal communication means 815 for communicating information, and a processor 810 coupled to the bus 815 for processing information. The processor may include one or more central processing units, graphical processing units, multi-core processors, and/or other computer processors. The system further comprises a random access memory (RAM) or other volatile storage device 850 (referred to as memory), coupled to bus 815 for storing information and instructions to be executed by processor 810. Main memory 850 also may be used for storing temporary variables or other intermediate information during execution of instructions by processor 810. The system also comprises a read only memory (ROM) and/or static storage device 820 coupled to bus 815 for storing static information and instructions for processor 810, and a data storage device 825 such as a magnetic disk or optical disk and its corresponding disk drive. Data storage device 825 is coupled to bus 815 for storing information and instructions.

The system may further be coupled to a display device 870, such as a light emitting diode (LED) display or a liquid crystal display (LCD) coupled to bus 815 through bus 865 for displaying information to a computer user. An alphanumeric input device 875, including alphanumeric and other keys, may also be coupled to bus 815 through bus 865 for communicating information and command selections to processor 810. An additional user input device is cursor control device 880, such as a touchpad, mouse, a trackball, stylus, or cursor direction keys coupled to bus 815 through bus 865 for communicating direction information and command selections to processor 810, and for controlling cursor movement on display device 870.

Another device, which may optionally be coupled to computer system 800, is a communication device 890 for accessing other nodes of a distributed system via a network. The communication device 890 may include any of a number of commercially available networking peripheral devices such as those used for coupling to an Ethernet, token ring, Internet, or wide area network. The communication device 890 may further be a null-modem connection, or any other mechanism that provides connectivity between the computer system 800 and the outside world. Note that any or all of the components of this system illustrated in FIG. 8 and associated hardware may be used in various embodiments as discussed herein.

It will be appreciated by those of ordinary skill in the art that any configuration of the system may be used for various purposes according to the particular implementation. The control logic or software implementing the described embodiments can be stored in main memory 850, mass storage device 825, or other storage medium locally or remotely accessible to processor 810.

It will be apparent to those of ordinary skill in the art that the system, method, and process described herein can be implemented as software stored in main memory 850 or read only memory 820 and executed by processor 810. This control logic or software may also be resident on an article of manufacture comprising a computer readable medium having computer readable program code embodied therein and being readable by the mass storage device 825 and for causing the processor 810 to operate in accordance with the methods and teachings herein.

The embodiments discussed herein may also be embodied in a handheld or portable device containing a subset of the computer hardware components described above. For example, the handheld device may be configured to contain only the bus 815, the processor 810, and memory 850 and/or 825. The handheld device may also be configured to include a set of buttons or input signaling components with which a user may select from a set of available options. The handheld device may also be configured to include an output apparatus such as a liquid crystal display (LCD) or display element matrix for displaying information to a user of the handheld device. Conventional methods may be used to implement such a handheld device. The implementation of embodiments for such a device would be apparent to one of ordinary skill in the art given the disclosure as provided herein.

The embodiments discussed herein may also be embodied in a special purpose appliance including a subset of the computer hardware components described above. For example, the appliance may include a processor 810, a data storage device 825, a bus 815, and memory 850, and only rudimentary communications mechanisms, such as a small touchscreen that permits the user to communicate in a basic manner with the device. In general, the more special purpose the device is, the fewer of the elements need be present for the device to function.

FIG. 9 is block diagram of one embodiment 900 of a mobile device. Mobile device 910 may be used, for example, as a user device when interacting with the care management platform.

In one embodiment, mobile device 910 is a system, which may include one or more processors 912, a memory 905, I/O controller 925, network interface 904, and display 920. Mobile device 910 may also include a number of processing modules, which may be implemented as hardware, software, firmware, or a combination. It should be appreciated that mobile device 910 may also include, although not illustrated, a user interface (e.g., keyboard, touchscreen, or similar devices), a power device (e.g., a battery), as well as other components typically associated with electronic devices. Network interface 904 may also be coupled to a number of wireless subsystems 915 (e.g., Bluetooth, Wi-Fi, Cellular, or other networks) to transmit and receive data streams through a wireless link to/from a network, or may be a wired interface for direct connection to networks (e.g., the Internet, Ethernet, or other wireless systems). In one embodiment, both network interface 904 and wireless subsystem 915 couple mobile device 910 to a network.

Memory 905 may be coupled to processor 912 to store instructions for execution by processor 912. In some embodiments, memory 905 is non-transitory. It should be appreciated that embodiments as described herein may be implemented through the execution of instructions, for example as stored in the memory 905 or other element, by processor 912 of mobile device 910 and/or other circuitry of mobile device 910 and/or other devices. Particularly, circuitry of mobile device 910, including but not limited to processor 912, may operate under the control of a program, routine, or the execution of instructions to execute methods or processes in accordance with the embodiments described herein. For example, such a program may be implemented in firmware or software (e.g., stored in memory 905 and/or other locations) and may be implemented by processors, such as processor 912, and/or other circuitry of mobile device 910. Further, it should be appreciated that the terms processor, microprocessor, circuitry, controller, etc., may refer to any type of logic or circuitry capable of executing logic, commands, instructions, software, firmware, functionality and the like.

Further, it should be appreciated that some or all of the functions, engines or modules described herein may be performed by mobile device 910 itself and/or some or all of the functions, engines or modules described herein may be performed by another system connected through I/O controller 925 or network interface 904 (wirelessly or wired) to mobile device 910. Thus, some and/or all of the functions may be performed by another system and the results or intermediate calculations may be transferred back to mobile device 910. In some embodiments, such other device may comprise a server, such as commerce platform 110 or 210.

It is to be understood that the above description is intended to be illustrative, and not restrictive. Many other embodiments will be apparent to those of skill in the art upon reading and understanding the above description. The scope should, therefore, be determined with reference to the appended claims, along with the full scope of equivalents to which such claims are entitled.

The foregoing description, for purpose of explanation, has been described with reference to specific embodiments. However, the illustrative discussions above are not intended to be exhaustive or to limit the described embodiments to the precise forms disclosed. Many modifications and variations are possible in view of the above teachings. The embodiments were chosen and described in order to best explain the principles and practical applications of the various embodiments, to thereby enable others skilled in the art to best utilize the various embodiments with various modifications as may be suited to the particular use contemplated.

What is claimed is:

1. A method for a care management platform and a user device interacting during the providing of real-time provider intelligence information, the method comprising:
receiving, by the care management platform from the user device, a request for patient records relevant to a patient, the request comprising a patient identifier (ID), a care provider identifier (ID), and a care provider context;
querying, by the care management platform, a patient data store for patient records based on the patient ID, care provider ID, and care provider context;
performing, by the care management platform, a first filtering of patient records returned in response to the querying, wherein the first filtering reduces the returned patient records to a first reduced set of patient records;
performing, by the care management platform, a second filtering of patient records in the first reduced set of patient records to generate a final reduced set of patient records; and
transmitting, by the care management platform to the user device, a response comprising at least a subset of the final reduced set of patient records.

2. The method of claim 1, wherein the first filtering comprises a rule-based filtering of the patient records returned in the response the querying, and wherein the second filtering comprises a machine learning (ML) based filtering of the patient records in the first reduced set of patient records, further comprising:
scoring, by an ML model, a relevancy of each of the patient records in the first reduced set of patient records based at least in part on the care provider context and zero or more unified medical language system (UMLS) concept unique identifiers (CUIs) mapped to one or more features of said each of the patient records; and
selecting the final reduced set of patient records based on the scores associated with said each of the patient records.

3. The method of claim 2, wherein the final reduced set of patient records comprises a subset of the first reduced set of patient records, and each member selected for the subset is associated with a relevancy score satisfying a relevancy threshold.

4. The method of claim 2, wherein the final reduced set of patient records comprises a subset of the first reduced set of patient records selected for having highest scores in the scoring of said each of the patient records.

5. The method of claim 2, further comprising:
receiving, by the care management platform from the user device, user feedback regarding a patient record from the final reduced set of patient records, the feedback comprising positive or negative feedback of the relevancy of the patient record to the care provider context;
adding, by the care management platform, the feedback for the patient with the patient record and the provider context to an ML retraining data set for the ML model;
periodically retraining, by the care management platform, the ML model to generate a tuned ML model; and
using, by the care management platform, the tuned ML model when scoring a relevancy of patient records associated with a second request for patient records.

6. The method of claim 5, wherein the user feedback is received by the care management platform from a graphical user interface rendered on the user device in response to a user selecting the patient record from within the graphical user interface, and wherein an application rendering the graphical user interface transmits the user feedback to the care management platform.

7. The method of claim 1, further comprising:
receiving, at the care management platform from the user device, a user query for additional patient records, the query comprising: a keyword or natural language query, a patient ID, a care provider ID, and a care provider context;
executing, by the care management platform, the query against the patient data store for the additional patient records based on the patient ID, provider ID, and provider context;
selecting, by the care management platform, a subset of the additional patient records; and
transmitting, by the care management platform to the user device, the selected subset of the additional patient records causing a user interface of the use device to display the selected subset of the additional patient records.

8. The method of claim 7, wherein the selecting comprises:
extracting, from each of the additional patient records and the user query, a semantic meaning of one or more features within each of the additional patient records and the user query;
constructing a semantic meaning vector for each of the additional patient records and the user query, the semantic meaning vector comprising a set of semantic meaning features and wherein extracted values are added to corresponding ones of the set of semantic meaning features in the semantic meaning vector constructed for said each of the additional patient records and the user query;
determining, by one or more ML model(s), a relevance score of a semantic meaning vector constructed for the user query to each semantic meaning vector corresponding to each of the additional patient records; and
selecting the subset of additional patient records as the additional patient records having a determined relevance score that satisfies a relevancy threshold.

9. The method of claim 1, wherein prior to receipt of the request, the method further comprises:
obtaining, by the care management platform, a new patient record, the new patient record being associated with the patient ID;
performing, by the care management platform, text extraction to extract one or more portions of text from the new patient record;
mapping, by the care management platform, zero or more unified medical language system (UMLS) concept unique identifiers (CUIs) to each of the one or more portions of text extracted from the new patient record, wherein each mapping is based on a matching of terms in a CUI to one or more terms in a portion of extracted text; and
filtering, by the care management platform, one or more irrelevant portions of text; and
adding, by the care management platform, the new patient record and one or more retained portions of text as new entries to the patient data store, wherein each new entry comprises metadata for the patient ID and metadata for the zero or more UMLS CUIs mapped to said each new entry.

10. The method of claim 9, wherein the new patient record is unstructured data and comprises an image of a patient record, and performing the text extraction comprises:
performing one or more image processing operations to extract text from the image of the patient record.

11. The method of claim 9, wherein the new patient record is structure data and comprises an electronic medical record, and performing the text extraction comprise:
extracting text from one or more text fields of the electronic medical record.

12. The method of claim 9, wherein filtering the one or more irrelevant portions of text, comprises:
applying a machine learning (ML) model trained to determine a relevancy of textual content and/or one or more UMLS CUIs associated with the textual content to a domain of care to be provided to the patient.

13. The method of claim 1, wherein the request is received as an application programming interface (API) based message generated by the user device, and the request is received at an API endpoint of the care management platform.

14. The method of claim 1, wherein the request and the response are transmitted using a secure protocol for the exchange of information over a communications network.

15. A non-transitory computer readable storage medium including instructions that, when executed by a processor, cause the processor to perform operations for a care management platform and a user device interacting during the providing of real-time provider intelligence information, the operations comprising:
receiving, by the care management platform from the user device, a request for patient records relevant to a patient, the request comprising a patient identifier (ID), a care provider identifier (ID), and a care provider context;
querying, by the care management platform, a patient data store for patient records based on the patient ID, care provider ID, and care provider context;
performing, by the care management platform, a first filtering of patient records returned in response to the querying, wherein the first filtering reduces the returned patient records to a first reduced set of patient records;
performing, by the care management platform, a second filtering of patient records in the first reduced set of patient records to generate a final reduced set of patient records; and
transmitting, by the care management platform to the user device, a response comprising at least a subset of the final reduced set of patient records.

16. The non-transitory computer readable storage medium of claim 15, wherein the first filtering comprises a rule-based filtering of the patient records returned in the response the querying, and wherein the second filtering comprises a machine learning (ML) based filtering of the patient records in the first reduced set of patient records, further comprising:
scoring, by an ML model, a relevancy of each of the patient records in the first reduced set of patient records based at least in part on the care provider context and zero or more unified medical language system (UMLS) concept unique identifiers (CUIs) mapped to one or more features of said each of the patient records; and
selecting the final reduced set of patient records based on the scores associated with said each of the patient records.

17. The non-transitory computer readable storage medium of claim 16, wherein the operations further comprise:
receiving, by the care management platform from the user device, user feedback regarding a patient record from the final reduced set of patient records, the feedback comprising positive or negative feedback of the relevancy of the patient record to the care provider context;
adding, by the care management platform, the feedback for the patient with the patient record and the provider context to an ML retraining data set for the ML model;
periodically retraining, by the care management platform, the ML model to generate a tuned ML model; and
using, by the care management platform, the tuned ML model when scoring a relevancy of patient records associated with a second request for patient records.

18. The non-transitory computer readable storage medium of claim 15, wherein the operations further comprise:
receiving, at the care management platform from the user device, a user query for additional patient records, the query comprising: a keyword or natural language query, a patient ID, a care provider ID, and a care provider context;
executing, by the care management platform, the query against the patient data store for the additional patient records based on the patient ID, provider ID, and provider context;
selecting, by the care management platform, a subset of the additional patient records; and
transmitting, by the care management platform to the user device, the selected subset of the additional patient records causing a user interface of the use device to display the selected subset of the additional patient records.

19. The non-transitory computer readable storage medium of claim 15, wherein prior to receipt of the request, the operations further comprise:
obtaining, by the care management platform, a new patient record, the new patient record being associated with the patient ID;
performing, by the care management platform, text extraction to extract one or more portions of text from the new patient record;
mapping, by the care management platform, zero or more unified medical language system (UMLS) concept unique identifiers (CUIs) to each of the one or more portions of text extracted from the new patient record, wherein each mapping is based on a matching of terms in a CUI to one or more terms in a portion of extracted text; and
filtering, by the care management platform, one or more irrelevant portions of text; and
adding, by the care management platform, the new patient record and one or more retained portions of text as new entries to the patient data store, wherein each new entry comprises metadata for the patient ID and metadata for the zero or more UMLS CUIs mapped to said each new entry.

20. A care management platform, comprising:
a memory; and
a processor coupled with the memory configured to:
receive, from a user device, a request for patient records relevant to a patient, the request comprising a patient identifier (ID), a care provider identifier (ID), and a care provider context,
query a patient data store for patient records based on the patient ID, care provider ID, and care provider context,
performing a first filtering of patient records returned in response to the querying, wherein the first filtering reduces the returned patient records to a first reduced set of patient records,
perform a second filtering of patient records in the first reduced set of patient records to generate a final reduced set of patient records, and
transmit, to the user device, a response comprising at least a subset of the final reduced set of patient records.
